(19) United States
(12) Patent Application Publication
(10) Pub. No.: US 2005/0192230 A1
(43) Pub. Date: Sep. 1, 2005

(54) Process for preparing 1- [N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine

(75) Inventors: Guenter Linz, Mittelbiberach (DE); Wolfgang Broeder, Heidesheim (DE); Georg Zerban, Ingelheim (DE); Eric Winter, Gau-Algesheim (DE); Bernd Schmitt, Ingelheim (DE); Svenja Renner, Ingelheim (DE); Bjoern Weyell, Aspisheim (DE); Heinz-Peter Schmitt, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GambH, Ingelheim (DE)

(21) Appl. No.: 11/040,273

(22) Filed: Jan. 20, 2005

Related U.S. Application Data
(60) Provisional application No. 60/548,690, filed on Feb. 27, 2004.

(30) Foreign Application Priority Data
Feb. 12, 2004 (DE) 102004006893

(51) Int. Cl. A61K 38/04; C07K 5/04

(52) U.S. Cl. 514/19; 514/252.17; 544/284

(57) ABSTRACT

The present application relates to a process for preparing the CGRP-antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine of formula 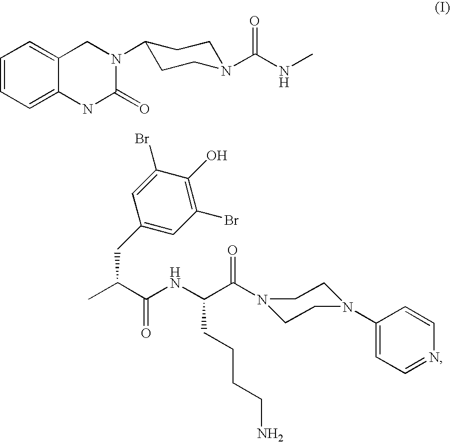 by means of which this compound can be prepared in large amounts, in high yields and with high purity.

The present application relates to a process for preparing the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine of formula 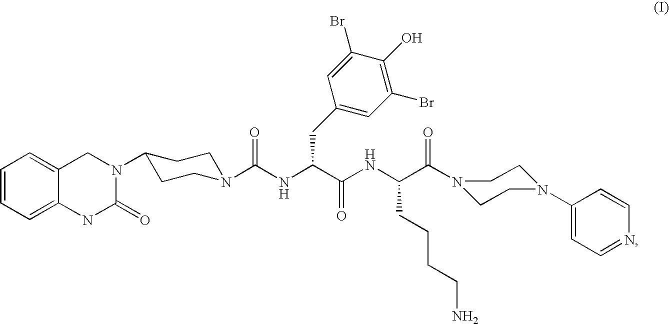
by means of which this compound can be prepared in large quantities, in high yields and with high purity.

### BACKGROUND OF THE INVENTION

The compound 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (I) is known from International Patent Application PCT/EP/9704862 (published as WO 98/11128) and constitutes a highly effective CGRP-antagonist for the acute and prophylactic treatment of headaches, particularly migraine and cluster headache. WO 98/11128 has already described a method of preparing quantities of the compound of formula I in grams, comprising chromatographic purification, which is not practicable for preparing large quantities.

In addition, it has been found that the compound of formula I is highly prone to oxidation, which leads to serious losses during manufacture.

The aim of the present invention was thus to develop an improved method of preparation which can also be used on an industrial or production scale. In particular, there should be no need for chromatographic purification in the new process. Moreover, the yields should be considerably higher at every stage and the product quality (by-product profile, purity, content) should be significantly improved.

### DESCRIPTION OF THE INVENTION

Surprisingly, it has been found that oxygen-free operation at every stage of the synthesis significantly increases both the yield and the purity of the intermediate products and hence also of the end product.

Therefore, to prepare the CGRP-antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine of formula (I), in a first step 1.0 to 1.2 equivalents, preferably 1.1 equivalents of an oxygen-free solution of the compound 3,4-dihydro-3-(4-piperidinyl)-2(1H)-quinazoline of formula 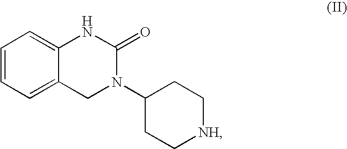
are reacted with 1.0 equivalents of a 3,5-dibromo-D-tyrosine ester of general formula 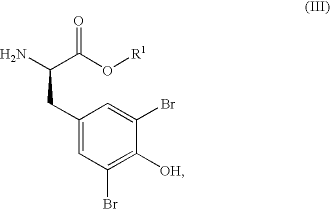
wherein R¹ denotes a straight-chain or branched C₁₋₆-alkyl or benzyl group, preferably a straight-chain or branched C₁₋₃-alkyl group, in the presence of 1.0 to 1.2 equivalents, preferably 1.1 equivalents, of a condensation agent such as carbonyldiimidazole, carbonylditriazole, 1,2,4-triazole, n-propanephosphonic anhydride, dicyclohexylcarbodiimide or 1-ethyl-3-(3′-dimethylaminopropyl)-carbodiimide, while the combination of carbonyldiimidazole and 1,2,4-triazole is preferably used, at temperatures between 0° C. and 60° C., preferably between 10° C. and ambient temperature. Suitable solvents are polar aprotic organic solvents such as tetrahydrofuran, dimethylformamide or N-methylpyrrolidinone, of which dimethylformamide is preferably used.

For further purification, the resulting 3,5-dibromo-N-[[4-(1,4-dihydro-2-oxo-3(2H)-quinazolinyl)-D-tyrosine-1-piperidinyl]carbonyl]-ester of general formula 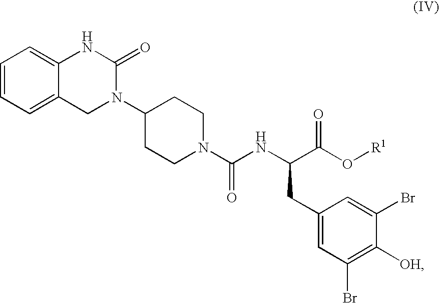
wherein R¹ denotes a straight-chain or branched C₁₋₆-alkyl or benzyl group, preferably a C₁₋₃-alkyl group, is suspended in an oxygen-free solvent, for example methanol, ethanol, isopropanol or mixtures thereof in a ratio of 1:0.9 to 1:1.2, preferably 1:1.1, the suspension is heated to temperatures of 70° to 80° C., then cooled to 0° C. and the solid is isolated.

The purified ester of general formula IV is saponified in a second step. For this purpose an oxygen-free aqueous or alcoholic solution of 2.0 to 3.0 equivalents, preferably 2.5 equivalents, of an alkali metal hydroxide, particularly lithium hydroxide, may be added dropwise. At the end of the reaction the solvent is separated off e.g. by distillation, so as to obtain the free acid 3,5-dibromo-N-[[4-(1,4-dihydro-2-oxo-3(2H)-quinazolinyl)-1-piperidinyl]carbonyl]-D-tyrosine of formula 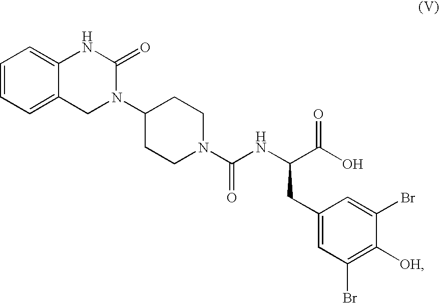
Suitable solvents other than water are alcohols such as methanol, ethanol or isopropanol.

In a third step the free acid of formula (V) obtained in the second step is reacted with an oxygen-free solution of a compound of general formula 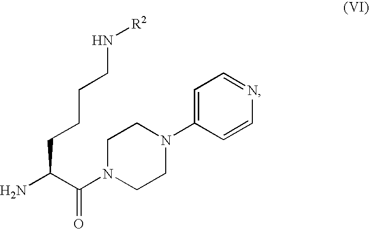
wherein R² denotes an amine protecting group, in a polar aprotic organic solvent such as e.g. tetrahydrofuran, dimethylformamide or N-methylpyrrolidinone, preferably dimethylformamide.

By amine protecting groups as specified in the definitions provided above and hereinafter are meant the protecting groups familiar from peptide chemistry, such as for example
a phenylalkoxycarbonyl group optionally substituted in the phenyl nucleus by a halogen atom, by a nitro or phenyl group or by one or two methoxy groups with 1 to 3 carbon atoms in the alkoxy moiety,
   for example the benzyloxycarbonyl, 2-nitro-benzyloxycarbonyl, 4-nitro-benzyloxycarbonyl, 4-methoxy-benzyloxycarbonyl, 2-chloro-benzyloxycarbonyl, 3-chloro-benzyloxycarbonyl, 4-chloro-benzyloxycarbonyl, 4-biphenylyl-α,α-di-methyl-benzyloxycarbonyl or 3,5-dimethoxy-α,α-dimethyl-benzyloxycarbonyl group,
an alkoxycarbonyl group with a total of 1 to 5 carbon atoms in the alkyl moiety,
   for example the methoxycarbonyl, ethoxycarbonyl, n-propoxycarbonyl, isopropoxycarbonyl, n-butoxycarbonyl, 1-methylpropoxycarbonyl, 2-methyl-propoxy-carbonyl or tert.butyloxycarbonyl group,
the allyloxycarbonyl, 2,2,2-trichloro-(1,1-dimethylethoxy)carbonyl or 9-fluorenyl-methoxycarbonyl group or
the formyl, acetyl or trifluoroacetyl group, while a straight-chain or branched C₁₋₈-alkyloxycarbonyl or a benzyloxycarbonyl group is preferably used and the tert.-butyloxycarbonyl group is most preferably used.

This reaction takes place in the presence of tertiary amines such as e.g. ethyldiisopropylamine, triethylamine or triisopropylamine, preferably in the presence of ethyldiisopropylamine, and a condensing agent. Suitable condensing agents are dicyclohexylcarbodiimide, carbonyldiimidazole, carbonylditriazole or 1-ethyl-3-(3′-dimethylaminopropyl)-carbodiimide, optionally in the presence of hydroxysuccinimide, hydroxybenzotriazole, p-nitrophenol or pentafluorophenol, which are used in excess. 1-ethyl-3-(3′-dimethylaminopropyl)-carbodiimide is particularly suitable.

The resulting compound of general formula 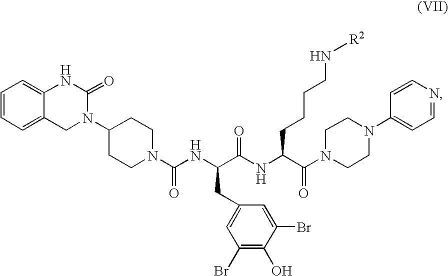
wherein R² denotes an amine protecting group, is converted into the compound of formula I in a fourth step by cleaving the amine protecting group under oxygen-free conditions in the usual way.

This process produces the compound of formula I in a total yield of 55% and with a purity of >99%.

In a first aspect the present invention thus relates to a process for preparing 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine of formula 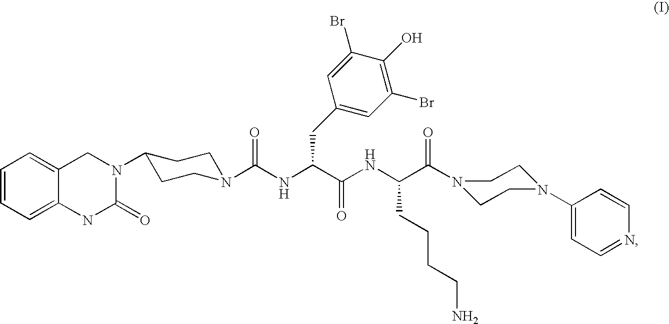
characterised in that
(a) 1.0 to 1.2 equivalents of an oxygen-free solution of the compound 3,4-dihydro-3-(4-piperidinyl)-2(1H)-quinazoline of formula 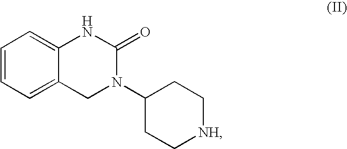
   are coupled with 1.0 equivalents of a 3,5-dibromo-D-tyrosine ester of general formula 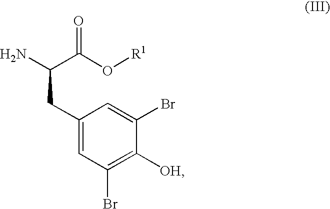
   wherein R¹ denotes a straight-chain or branched C₁₋₆-alkyl or benzyl group, preferably a straight-chain or branched C₁₋₃-alkyl group, in the presence of 1.0 to 1.2 equivalents of a condensation agent and the resulting 3,5-dibromo-N-[[4-(1,4-dihydro-2-oxo-3(2H)-quinazolinyl)-D-tyrosine-1-piperidinyl]carbonyl]-ester of general formula 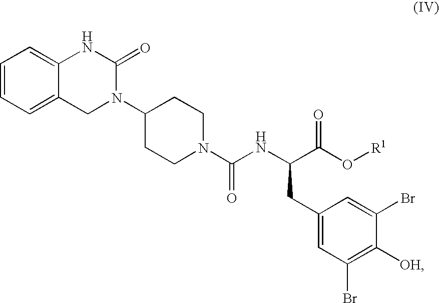
   wherein R¹ denotes a straight-chain or branched C₁₋₆-alkyl or benzyl group, preferably a straight-chain or branched C₁₋₃-alkyl group, is then crystallised under oxygen-free conditions,
(b) the 3,5-dibromo-N-[[4-(1,4-dihydro-2-oxo-3(2H)-quinazolinyl)-D-tyrosine-1-piperidinyl]carbonyl]-ester of general formula IV thus obtained is saponified by the dropwise addition of an oxygen-free aqueous or alcoholic solution of 2.0 to 3.0 equivalents of an alkali metal hydroxide, thus obtaining the free acid 3,5-dibromo-N-[[4-(1,4-dihydro-2-oxo-3(2H)-quinazolinyl)-1-piperidinyl]carbonyl-D-tyrosine of formula 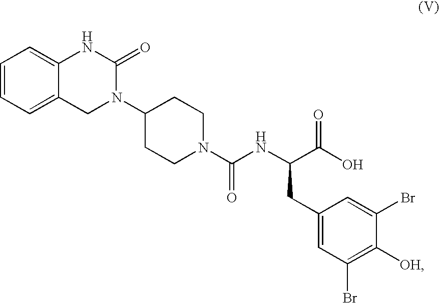
(c) the free acid of formula V is coupled with an oxygen-free solution of a compound of general formula 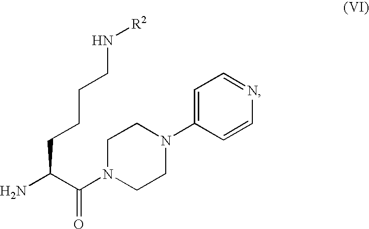
   wherein R² denotes an amine protecting group, and
(d) the amine protecting group of the resulting compound of general formula 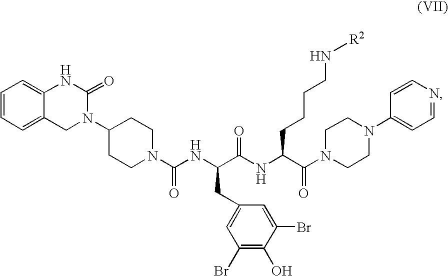
   wherein R² denotes an amine protecting group, is cleaved under oxygen-free conditions, thereby producing the compound of formula I according to the invention in pure form.
   Experimental Section

The following abbreviations are used:
CDI carbonyldiimidazole
DMF dimethylformamide
of th. of theoretical
eq. equivalent(s)
conc. concentrated
MtBE methyl-tert.-butylether
mp. melting point
TEAP
   Step 1:

### Preparation of 3,4-dihydro-3-[1-(phenylmethyl)-4-piperidinyl]-2(1H)-quinazoline

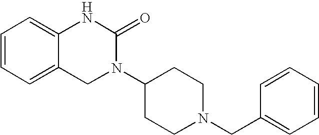

4-aminobenzylpiperidine (66.2 g, 0.35 mol, 1.05 eq.) are metered into an inertised solution of 2-nitrobenzaldehyde (50 g, 0.33 mol, 1.0 eq.) in methanol (240 ml) at 10-20° C. The solution is heated to 40-45° C. heated and stirred for about 1 hour. Then the mixture is cooled to 20-25° C. and adjusted to a pH of 9 with some conc. hydrochloric acid.

Conc. hydrochloric acid (chemically pure, approx. 37%, 12 ml) and a suspension prepared from sodium borohydride (7.1 g, 0.19 mol, 0.57 eq.), water (15 ml) and conc. sodium hydroxide solution (0.3 ml, chemically pure, about 45%) are metered into this solution simultaneously so that the selected pH remains constant (care needed, gas given off, exothermic reaction!) After this addition the mixture is stirred for about 1 hour, adjusted to pH 1-1.5 with conc. hydrochloric acid (chemically pure, approx. 37%, 65 ml) (destroying excess sodium borohydride!) and finally adjusted back to pH 9 with conc. sodium hydroxide solution (chemically pure, approx. 45%, 38 ml).

The solvent mixture is distilled off in vacuo at a maximum temperature of 70° C. The residue is suspended in DMF (330 ml), briefly dewatered at 60° C. in an oil pump vacuum and the suspension cooled to 5-10° C. is filtered to remove the precipitated salts (boron salts, common salt).

The solution (about 365 ml) is hydrogenated in the presence of Raney nickel (3.4 g, 50% moistened with water) at 40-60° C. and 2 bar.

After the catalyst has been filtered off 180-200 ml of DMF are distilled off at a maximum temperature of 80° C. under an oil pump vacuum (note: the water content should be less than 2% so as to prevent excess decomposition of the carbonyidiimidazole (CDI) used subsequently).

CDI (64.5 g, 0.4 mol, 1.2 eq.) is added batchwise to the solution cooled to 20-25° C., so as not to exceed a temperature of 35° C. After the addition and a brief period of stirring the suspension is cooled to 20-25° C. and a mixture of water (490 ml) and methanol (120 ml) is allowed to run into it. The crystals formed are filtered off at 0° C. and washed again with a water/methanol mixture (160 ml, 40 ml) and then dried in vacuo.

Yield: 85 g (80% of th., based on 2-nitrobenzaldehyde)

Physicochemical characterisation of 3,4-dihydro-3-[1-(phenylmethyl)-4-piperidinyl]-2(1H)-quinazoline:

M.P.: 205-206° C.

Purity: 99.7% HPLC peak area

Chromatographic Conditions:
Constant flow pump, gradient system, e.g. HP1090
injection system: automatic feed system, e.g. HP1090
injection volume 6 μl
column: 250×4.6 mm in diameter.
stationary phase: Hypersil BDS CPS; 3 μm

mobile phase: solvent A: 0.05M triethylammonium acetate pH=5.0 solvent B: methanol

| gradient | run time | solvent A | solvent B |
|---|---|---|---|
| | 0 min | 90% | 10% |
| | 5 min | 90% | 10% |
| | 30 min | 70% | 30% |
| | 40 min | 30% | 70% |
| | 55 min | 10% | 90% |

equilibration time: 8 minutes
run time: 55 minutes
oven temperature: 45° C.
throughflow: 0.7 ml/minute
detector: UV-detector, e.g. DAD, HP1040, cell 10 mm
measuring wavelength: 250 nm
   Step 2:

### Preparation of 3,4-dihydro-3-(4-piperidinyl)-2(1H)-quinazoline

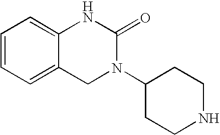 3,4-dihydro-3-[1-(phenylmethyl)-4-piperidinyl]-2(1H)-quinazoline (52 g, 0.16 mol, 1.0 eq.) is dissolved in ethanol (260 ml) and hydrogenated in the presence of palladium/charcoal (20.8 g, 50% moistened with water) at 60° C. and 2 bar. After the catalyst has been filtered off the solvent mixture (ethanol, toluene) is distilled off in vacuo at a maximum temperature of 70° C. and the residue is combined first with methanol (52 ml), then with MtBE (170 ml) and the mixture is refluxed for at least 30 minutes. The substance crystallises from the solution which has been cooled to below 0° C., is filtered off, washed with MtBE (70 ml) and dried in vacuo.

Yield: 31.5 g (85% of th.)

Physicochemical characterisation of 3,4-dihydro-3-(4-piperidinyl)-2(1H)-quinazoline:
M.p.: 195° C.
Purity: 99.7% HPLC peak area,

Chromatographic Conditions:
Constant flow pump, gradient system, e.g. HP1090
injection system: automatic feed system, e.g. HP1090
injection volume: 5 μl
column: 250×4.6 mm in diameter.
stationary phase: Nucleosil C18 AB; 5 μm

mobile phase: solvent A: 0.5% KH₂PO₄, pH 3.0 solvent B: methanol/acetonitrile 50/50

| gradient | run time | solvent A | solvent B |
|---|---|---|---|
| | 0 min | 90% | 10% |
| | 15 min | 90% | 10% |
| | 30 min | 50% | 50% |
| | 45 min | 20% | 80% |

equilibration time: 8 minutes
run time: 45 minutes
oven temperature: ambient temperature
throughflow: 0.8 ml/minute
detector: UV-detector, e.g. DAD, HP1040, cell 10 mm
measuring wavelength: 254 nm
   Step 3:

### Preparation of [(5S)-5-amino-6-oxo-6-[4-(4-pyridinyl)-1-piperazinyl]hexyl]-1,1-di-methylethylester-mono[tetrafluoroborate(1-)]-carbamoyl acid, crude

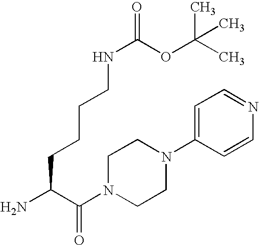 A solution of 1-(4-pyridyl)-piperazine (8.05 g, 49 mmol, 1.05 eq.) in DMF (25 ml) is metered within 30 minutes into a solution of Z-Lys(Boc)-OSu (22.32 g, 46 mmol, 1.0 eq.) in DMF (37 ml) and the mixture is stirred for another 4 hours. Then toluene (270 ml) and sodium hydrogen carbonate solution (4.5 g to 65 ml of water) are added and the organic phase is separated off. The aqueous phase is extracted twice more with toluene (270 ml) and then discarded. The product phase is washed first with sodium hydrogen carbonate solution (3×23 ml) and with water (2×10 ml) and the solvent is distilled off. The residue is taken up with methanol (200 ml) and hydrogenated in the presence of palladium/charcoal (1.67 g, 50% moistened with water) at 40° C. and 4 bar. After filtration of the catalyst the solution is concentrated (˜⅛) and cooled to 0 to 4° C. Ammonium tetrafluoroborate (5.3 g, 50 mmol, 1.1 eq.) dissolved in water (19 ml) is added dropwise to this solution. The substance then begins to crystallise out. It is stirred for another 1 hour and then filtered. The crystals are washed first with cold methanol (56 ml), then with MtBE (56 ml) and dried in vacuo.

Yield: 17.6 g (80% of th., based on Z-Lys(Boc)-OSu)

Physicochemical characterisation of [(5S)-5-amino-6-oxo-6-[4-(4-pyridinyl)-1-pipera-zinyl]hexyl]-1,1-dimethylethylester-mono[tetrafluoroborate(1-)]carbamoyl acid, crude:
M.p.: 208° C.
Purity: 99.5% HPLC peak area,

Chromatographic Conditions:
Constant flow pump, gradient system, e.g. HP1090
injection system: automatic feed system e.g. HP1090
injection volume: 10 μl
column: 250×4.6 mm in diameter.
stationary phase: Kromasil 100 C18; 5 μm

mobile phase:
solvent A: 0.1% 0.01 M octanesulphonic acid-sodium salt in triethylammonium phosphate buffer, pH 3.0

solvent B: 0.01 M octanesulphonic acid-sodium salt in acetonitrile

| gradient | run time | solvent A | solvent B |
|---|---|---|---|
| | 0 min | 70% | 30% |
| | 3 min | 70% | 30% |
| | 15 min | 65% | 35% |
| | 45 min | 40% | 60% |
| | 50 min | 30% | 70% |
| | 52 min | 30% | 70% |

equilibration time: 8 minutes
run time: 52 minutes
oven temperature: ambient temperature
throughflow: 1.0 ml/minute
detector: UV-detector, e.g. DAD, HP1040, cell 10 mm
measuring wavelength: 210 nm
   Step 4:

### Purification of [(5S)-5-amino-6-oxo-6-[4-(4-pyridinyl)-1-piperazinyl]hexyl]-1,1-dimethylethylester-mono[tetrafluoroborate(1-)]carbamoyl acid

The crude [(5S)-5-amino-6-oxo-6-[4-(4-pyridinyl)-1-piperazinyl]hexyl]-1,1-dimethyl-ethylester-mono[tetrafluoroborate(1-)]carbamoyl acid from Step 3 (16 g, 33.3 mmol) is suspended in ethanol (68 ml) and refluxed. Water (40 ml) is added dropwise to this hot solution. The solution is filtered hot, cooled to 0-4° C. and the precipitated crystals are filtered off. They are washed first with cold ethanol/water mixture (4+2.5 ml), then with cold ethanol (7 ml) and the substance is dried in vacuo.

Yield: 15.2 g (95% of th.)

Physicochemical characterisation of [(5S)-5-amino-6-oxo-6-[4-(4-pyridinyl)-1-pipera-zinyl]hexyl]-1,1-dimethylethylester-mono[tetrafluoroborate(1-)]carbamoyl acid, pure:
M.p.: 208° C.
Purity: 99.9% HPLC peak area,

Chromatographic Conditions:
Constant flow pump, gradient system, e.g. HP1090
injection system: automatic feed system, e.g. HP1090
injection volume: 10 μl
column: 250×4.6 mm in diameter.
stationary phase: Kromasil 100 C18; 5 μm

mobile phase:
solvent A: 0.1% 0.01M octanesulphonic acid-sodium salt in triethylammonium phosphate buffer, pH 3.0

solvent B: 0.01 M octanesulphonic acid-sodium salt in acetonitrile

| gradient | run time | solvent A | solvent B |
|---|---|---|---|
| | 0 min | 70% | 30% |
| | 3 min | 70% | 30% |
| | 15 min | 65% | 35% |
| | 45 min | 40% | 60% |
| | 50 min | 30% | 70% |
| | 52 min | 30% | 70% |

equilibration time: 8 minutes
run time: 52 minutes
oven temperature: ambient temperature
throughflow: 1.0 ml/minute
detector: UV-detector, e.g. DAD, HP1040, cell 10 mm
measuring wavelength: 210 nm
   Step 5:

### Preparation of 3,5-dibromo-N-[[4-(1,4-dihydro-2-oxo-3(2H)-quinazolinyl)-D-tyrosine-1-piperidinyl]carbonyl]-methylester, crude

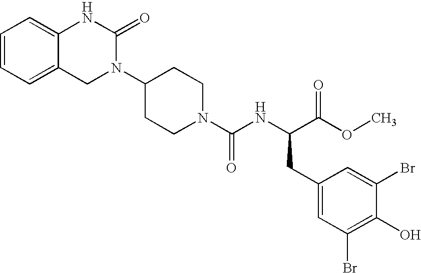

3,5-dibromo-D-tyrosine-methylester (10.0 g, 28.3 mmol, 1.0 eq.) and 1,2,4-triazole (4.52 g, 65.4 mmol, 2.3 eq.) are suspended in oxygen-free DMF (70 ml) at 10° C. Then 1,1-carbonyldiimidazole (5.05 g, 31.1 mmol, 1.1 eq.) is added batchwise and the mixture is stirred for 1 hour at 10° C. and for 1 hour at ambient temperature. The reaction solution thus prepared is added dropwise to a solution of 3,4-dihydro-3-(4-piperidinyl)-2(1H)-quinazoline (7.21 g, 31.2 mmol, 1.1 eq.) in oxygen-free DMF (150 ml) and then heated to 50-55° C. and stirred for 1.5 hours. The mixture is cooled to ambient temperature and within 1 hour added dropwise to oxygen-free water (550 ml). The white suspension is stirred overnight at ambient temperature and then filtered off. The residue is washed with oxygen-free water (4×50 ml) and dried in vacuo.

Yield: 15.2 g (88% of th.)

Physicochemical characterisation of 3,5-dibromo-N-[[4-(1,4-dihydro-2-oxo-3(2H)-quinazolinyl)-D-tyrosine-1-piperidinyl]carbonyl]-methylester, crude:
M.P.: 215° C.
Purity: 99.3% HPLC peak area

Chromatographic Conditions:
Constant flow pump, gradient system, e.g. HP1090
injection system: automatic feed system, e.g. HP1090
injection volume: 15 μl
column: 250×4.6 mm in diameter.
stationary phase: YMC ODS AM; 5 μm

mobile phase: solvent A: 0.1% KH₂PO₄, pH 3.0 solvent B: methanol/acetonitrile 50/50

| gradient | run time | solvent A | solvent B |
|---|---|---|---|
| | 0 min | 90% | 10% |
| | 10 min | 90% | 10% |
| | 25 min | 50% | 50% |
| | 50 min | 50% | 50% |
| | 75 min | 10% | 90% |

equilibration time: 8 minutes
run time: 75 minutes
oven temperature: ambient temperature
throughflow: 1.0 ml/minute
detector: UV-detector, e.g. DAD, HP1040, cell 10 mm
measuring wavelength: 215 nm
   Step 6:

### Purification of 3,5-dibromo-N-[[4-(1,4-dihydro-2-oxo-3(2H)-quinazolinyl)-D-tyrosine-1-piperidinyl]carbonyl]-methylester

Crude 3,5-dibromo-N-[[4-(1,4-dihydro-2-oxo-3(2H)-quinazolinyl)-D-tyrosine-1-piperidinyl]carbonyl]-methylester (10 g, 16.4 mmol) is suspended in oxygen-free ethanol (125 ml) and isopropanol (150 ml) and the mixture is refluxed for 20 minutes. Then within 5 hours it is cooled to 0° C. and stirred for 1 hour. The crystalline material is filtered off and washed with ice-cold oxygen-free isopropanol (2×20 ml). The drying is carried out in vacuo.

Yield: 8.79 g (88% of th.)

Physicochemical characterisation of 3,5-dibromo-N-[[4-(1,4-dihydro-2-oxo-3(2H)-quinazolinyl)-D-tyrosine-1-piperidinyl]carbonyl]-methylester, pure:
M.P.: 215° C.
Purity: 99.7% HPLC peak area,

Chromatographic Conditions:
Constant flow pump, gradient system, e.g. HP1090
injection system: automatic feed system, e.g. HP1090
injection volume 15 μl
column 250×4.6 mm in diameter.
stationary phase YMC ODS AM; 5 μm

mobile phase: solvent A: 0.1 % KH₂PO₄, pH 3.0 solvent B: methanol/acetonitrile 50/50

| gradient | run time | solvent A | solvent B |
|---|---|---|---|
| | 0 min | 90% | 10% |
| | 10 min | 90% | 10% |
| | 25 min | 50% | 50% |
| | 50 min | 50% | 50% |
| | 75 min | 10% | 90% |

equilibration time: 8 minutes
run time: 75 minutes
oven temperature: ambient temperature
throughflow: 1.0 ml/minute
detector: UV-detector, e.g. DAD, HP1040, cell 10 mm
measuring wavelength: 215 nm
   Step 7:

### Preparation of 3,5-dibromo-N-[[4-(1,4-dihydro-2-oxo-3(2H)-quinazolinyl)-1-piperidinyl]carbonyl]-D-tyrosine

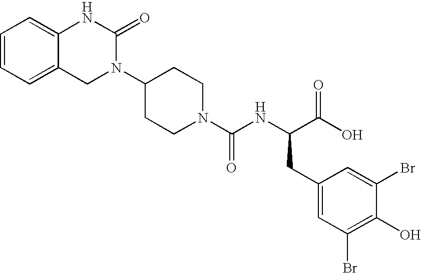

Pure 3,5-dibromo-N-[[4-(1,4-dihydro-2-oxo-3(2H)-quinazolinyl)-D-tyrosine-1-piperidinyl]carbonyl]-methylester (7.50 g, 12.9 mmol, 1.0 eq.) is suspended in oxygen-free water (50 ml) and combined with a solution of LiOH (0.74 g, 30.9 mmol, 2.5 eq.) in oxygen-free water (15 ml). The reaction mixture is stirred for 3 hours and then added dropwise to a mixture of oxygen-free DMF (8 ml), water (25 ml) and conc. HCl (3.3 ml, 32%). The suspension is stirred for 3 hours, the solid is filtered off and dried in vacuo. Yield: 7.30 g (99% of th.)

Physicochemical characterisation of 3,5-dibromo-N-[[4-(1,4-dihydro-2-oxo-3(2H)-quinazolinyl)-1-piperidinyl]carbonyl]-D-tyrosine:
M.p.: 210° C.
Purity: 99.8% HPLC peak area,

Chromatographic Conditions:
Constant flow pump, gradient system, e.g. HP1090
injection system: automatic feed system e.g. HP1090
injection volume: 10 μl
column: 250×4.6 mm in diameter.
stationary phase: YMC ODS AM; 5 μm

mobile phase: solvent A: 0.1% KH₂PO₄, pH 3.0 solvent B: methanol

| gradient | run time | solvent A | solvent B |
|---|---|---|---|
| | 0 min | 70% | 30% |
| | 5 min | 70% | 30% |
| | 15 min | 45% | 55% |
| | 40 min | 45% | 55% |
| | 55 min | 5% | 95% |
| | 60 min | 5% | 95% |

equilibration time: 8 minutes
run time: 60 minutes
oven temperature: ambient temperature
throughflow: 1.0 ml/minute
detector: UV-detector, e.g. DAD, HP1040, cell 10 mm
measuring wavelength: 290 nm
   Step 8:

### Preparation of [R—(R*,S*)]-[5-[[3-(3,5-dibromo-4-hydroxyphenyl)-2-[[[4-(1,4-dihydro-2-oxo-3(2H)-quinazolinyl)-1-piperidinyl]carbonyl]amino]-1-oxopropyl]amino]-6-oxo-6-[4-(4-pyridinyl)-1-piperazinyl]hexyl]-carbamoyl acid-1,1-dimethylethylester

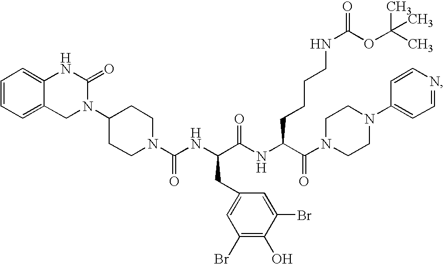

[(5S)-5-amino-6-oxo-6-[4-(4-pyridinyl)-1-piperazinyl]hexyl]-1,1-dimethylethylester-mono[tetrafluoroborate(1-)]carbamoyl acid (4.84 g, 10.1 mmol, 1.01 eq.) and N-ethyldiisopropylamine (1.55 g, 12.0 mmol, 1.2 eq.) are added to a solution of 3,5-dibromo-N-[[4-(1,4-dihydro-2-oxo-3(2H)-quinazolinyl)-1-piperidinyl]carbonyl]-D-tyrosine (5.96 g, 10.0 mmol, 1.0 eq.) in oxygen-free DMF (40 ml) and the mixture is cooled to 0° C. Then N-(3-dimethylaminopropyl)-N-ethylcarbonyldiimide hydrochloride (1.92 g, 10.02 eq.) is metered in within 2 hours in small batches. The mixture is then stirred for 12 hours at ambient temperature. The crude solution is added dropwise to a mixture of potassium hydrogen phosphate (8.79 g, 64.6 mmol, 6.5 eq.) in oxygen-free water (250 ml) and stirred for 1 hour at 40° C. and for another 12 hours at ambient temperature. The solid is filtered off, washed with water (4×60 ml) and dried in vacuo.

Yield: 8.05 g (83% of th.)

Physicochemical characterisation of [R—(R*,S*)]-[5-[[3-(3,5-dibrbmo-4-hydroxy-phenyl)-2-[[[4-(1,4-dihydro-2-oxo-3(2H)-quinazolinyl)-1-piperidinyl]carbonyl]amino]-1-oxopropyl]amino]-6-oxo-6-[4-(4-pyridinyl)-1-piperazinyl]hexyl]-carbamoyl acid-1,1-dimethylethylester:
M.p.: 209° C.
Purity: 98.7% HPLC peak area

Chromatographic Conditions:
Constant flow pump, gradient system, e.g. HP1090
injection system: automatic feed system e.g. HP1090
injection volume: 4 μl
column: 125×4.6 mm in diameter.
stationary phase: Inertsil ODS-2; 5 μm

mobile phase: solvent A: TEAP buffer, pH 3.0 solvent B: acetonitrile

| gradient | run time | solvent A | solvent B |
|---|---|---|---|
| | 0 min | 95% | 5% |
| | 3 min | 95% | 5% |
| | 15 min | 73% | 27% |
| | 30 min | 73% | 27% |
| | 35 min | 70% | 30% |
| | 40 min | 70% | 30% |
| | 65 min | 45% | 55% |

equilibration time: 10 minutes
run time: 65 minutes

1oven temperature: 45° C.
throughflow: 1.0 ml/minute
detector: UV-detector e.g. DAD, HP1040, cell 10 mm
measuring wavelength: 215 nm
   Step 9:

### Preparation of 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine

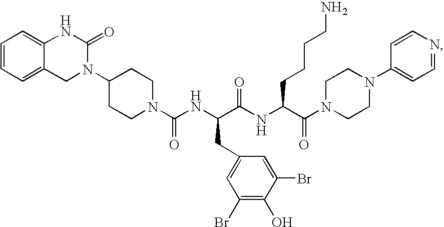

A solution of [R—(R*,S*)]-[5-[[3-(3,5-dibromo-4-hydroxyphenyl)-2-[[[4-(1,4-dihydro-2-oxo-3(2H)-quinazolinyl)-1-piperidinyl]carbonyl]amino]-1-oxopropyl]amino]-6-oxo-6-[4-(4-pyridinyl)-1-piperazinyl]hexyl]-carbamoyl acid-1,1-dimethylethylester (4.00 g, 4.00 mmol, 1.0 eq.) in oxygen-free DMF (10 ml) is slowly added dropwise to an ice-cooled, oxygen-free solution of HCl_{(g)} (4N) in 1,4-dioxane (60 ml) and the cloudy mixture is stirred for 2.5 hours at 12° C. To stop the reaction oxygen-free acetone (160 ml) and DMF (12 ml) are added and then the precipitate is filtered off and washed with oxygen-free acetone (200 ml). The residue is dissolved in oxygen-free water (120 ml) and combined with a mixture of ammonia (25%, 7 ml) in water (40 ml). The white suspension is stirred for 1 hour, the solid is separated off, washed with oxygen-free water (2×80 ml) and dried in vacuo.

Yield: 2.79 g (78% of th.)

Physicochemical characterisation of 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine:
M.p.: 203° C.
Purity: 99.3% HPLC peak area

Chromatographic Conditions:
Constant flow pump, gradient system, e.g. HP1090 or HP1100
injection system: automatic feed system e.g. HP1090
injection volume: 7 μl
column: 125×4.6 mm in diameter.
stationary phase: lnertsil ODS-2; 5 μm

mobile phase: solvent A: 0.5 % KH₂PO₄, pH=3.0 solvent B: acetonitrile

| gradient | run time | solvent A | solvent B |
|---|---|---|---|
| | 0 min | 100% | 0% |
| | 6 min | 88% | 12% |
| | 12 min | 81.5% | 18.5% |
| | 23 min | 81.5% | 18.5% |
| | 38 min | 45% | 55% |

run time: 38 minutes
equilibration time: 8 minutes
oven temperature: 45° C.
throughflow: 1.0 ml/minute
detector: UV-detector, e.g. DAD, HP1040, cell 10 mm
measuring wavelength: 215 nm

1. A process for preparing 1-[N²-[3,5-dibromo-N-[[4-(3,4-dihydro-2(1H)-oxo-quinazolin-3-yl)-1-piperidinyl]carbonyl]-D-tyrosyl]-L-lysyl]-4-(4-pyridinyl)-piperazine of formula 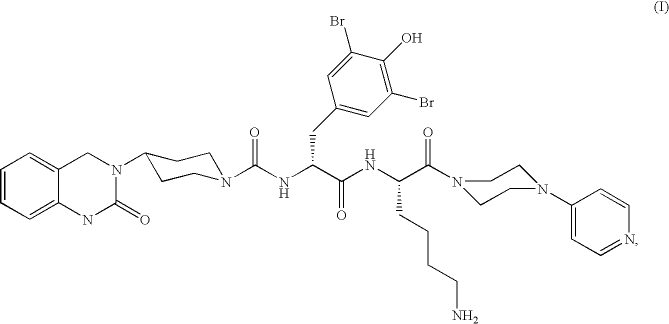
wherein
(a) 1.0 to 1.2 equivalents of an oxygen-free solution of the compound 3,4-dihydro-3-(4-piperidinyl)-2(1H)-quinazoline of formula 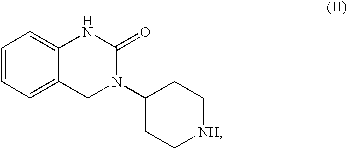
are coupled with 1.0 equivalents of a 3,5-dibromo-D-tyrosine ester of the formula 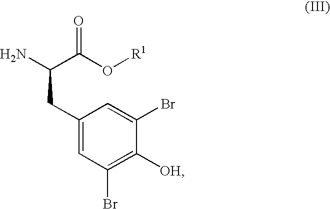
wherein R¹ denotes a straight-chain or branched C₁₋₆-alkyl or benzyl group, preferably a straight-chain or branched C₁₋₃-alkyl group, in the presence of 1.0 to 1.2 equivalents of a condensation agent and the resulting 3,5-dibromo-N-[[4-(1,4-dihydro-2-oxo-3(2H)-quinazolinyl)-D-tyrosine-1-piperidinyl]carbonyl]-ester of general formula 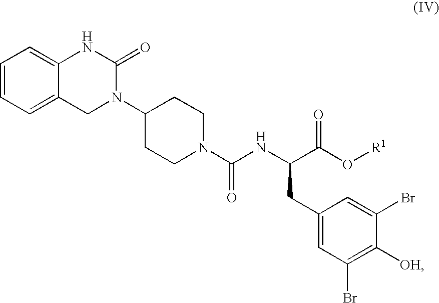
wherein R¹ denotes a straight-chain or branched C₁₋₆-alkyl or benzyl group, is then crystallised under oxygen-free conditions,
(b) the 3,5-dibromo-N-[[4-(1,4-dihydro-2-oxo-3(2H)-quinazolinyl)-D-tyrosine-1-piperidinyl]carbonyl]-ester of the formula IV thus obtained is saponified by the dropwise addition of an oxygen-free aqueous or alcoholic solution of 2.0 to 3.0 equivalents of an alkali metal hydroxide, thus producing the free acid 3,5-dibromo-N-[[4-(1,4-dihydro-2-oxo-3(2H)-quinazolinyl)-1-piperidinyl]carbonyl-D-tyrosine of formula 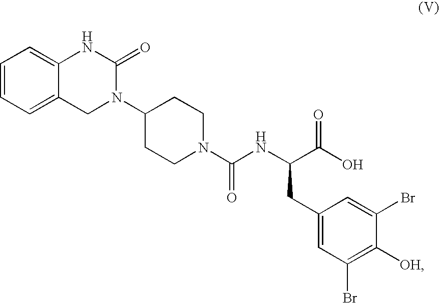
(c) the free acid of formula V is coupled with an oxygen-free solution of a compound of the formula 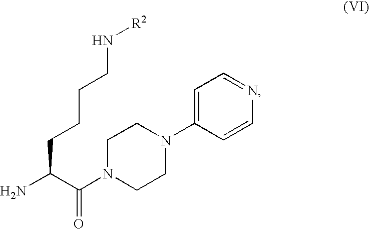
wherein R² denotes an amine protecting group, and
(d) the amine protecting group of the resulting compound of the formula 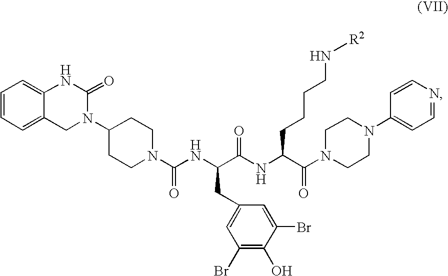
wherein R² denotes an amine protecting group, is cleaved under oxygen-free conditions, as a result of which the compound of formula I according to the invention is obtained in pure form.
